(19) United States
(12) Patent Application Publication
(10) Pub. No.: US 2018/0000743 A1
(43) Pub. Date: Jan. 4, 2018

(54) Modified Alginate Hydrogels for Therapeutic Agents, their Preparation and Methods Thereof

(71) Applicants: Wake Forest University, Winston-Salem, NC (US); Wake Forest University Health Sciences, Winston-Salem, NC (US)

(72) Inventors: Mark E. Welker, Clemmons, NC (US); Emmanuel C. Opara, Durham, NC (US); Surya Banks, Winston-Salem, NC (US)

(21) Appl. No.: 15/606,769

(22) Filed: May 26, 2017

Related U.S. Application Data
(60) Provisional application No. 62/357,741, filed on Jul. 1, 2016.

(51) Int. Cl. A61K 9/50; A61K 31/4458; A23P 10/30; A61K 35/747; A61K 31/4525; A23L 33/00; A61K 31/4184; A61K 31/235; C08B 37/00; A61K 31/485; A61K 35/00

(52) U.S. Cl. CPC A61K 9/5036; C08B 37/0084; A61K 31/4458; A23P 10/30; A61K 35/747; A61K 31/485; A61K 31/4525; A23L 33/30; A61K 31/4184; A61K 31/235; A23V 2002/00; A61K 2035/115

(57) ABSTRACT

A novel chemically modified alginate hydrogel has been developed which combines an aromatic compound with a carbohydrate, where the aromatic compound is one or more amines combined with an alginate. The chemical structure of alginate is modified using different amines and different methods, including: (1) covalently bonding aminoethyl benzoic acid to the alginate backbone, and (2) oxidizing the vicinal diol in the alginate chain to an aldehyde before coupling to aminoethyl benzoic acid. Alternatively, the combined aromatic compound and carbohydrate can be a dopamine combined with the alginate. The chemically modified alginate and the methods used can be utilized to encapsulate a variety of bioactive substances for oral delivery in humans and animals, including, but not limited to: (i) drugs, medicines, enzymes, proteins, hormones, and vaccines, (ii) vitamins, minerals, micronutrients and/or other dietary supplements, (iii) probiotics and/or other microorganisms, (iv) cells, cell parts, and/or other biological materials, and/or (v) other bioactive substances.

The present application claims priority under 35 USC 119(e) to U.S. Provisional Application No. 62/357,741 filed Jul. 1, 2016, the entire contents of which are incorporated by reference.

### FIELD OF THE INVENTION

The present invention relates to modified alginate hydrogels, methods of preparing these hydrogels, and their methods of use.

### BACKGROUND OF THE INVENTION

Certain therapeutic and bioactive substances, including, but not limited to, (i) medicines, drugs, enzymes, proteins, hormones, and vaccines, (ii) vitamins, minerals, micronutrients and other dietary supplements, (iii) probiotics and other micro-organisms, (iv) cells, cell parts, and/or biological materials, and/or (v) many other bioactive substances have been found to be vital, therapeutic and necessary for, among other things, the treatment, prevention, and/or inhibition of certain diseases and other conditions in humans and animals, the elimination or reduction of pain associated with a wide variety illnesses, diseases and conditions, and the general maintenance of good health and well-being in humans and animals, including pets and livestock. For many of these substances, the simplest and most cost-effective method for delivering the medicine or other substance to humans and animals is by oral delivery in the form of a pill, capsule, liquid, paste, or other currently available oral delivery method.

Current oral delivery methods, however, suffer from a number of significant drawbacks and limitations, depending on the substance being delivered. Chief among these is the fact that many substances taken orally are attacked, degraded and/or destroyed in the stomach by stomach acids and/or enzymatic action. Insulin, for example (a vital and necessary protein/hormone), is destroyed in the stomach by stomach acids and enzymatic action. This is the primary reason insulin cannot currently be administered orally. Rather, insulin is currently administered by injection or intravenously, adding substantial costs and other problems to the delivery of this vital hormone, which is needed by millions of people around the world on a daily basis. There are numerous other examples of other therapeutic proteins, hormones, and other bioactive substances that are destroyed or rendered ineffective by stomach acids and/or enzymatic action. This problem would benefit greatly from a viable and cost-effective oral delivery solution.

Even if a substance is not completely destroyed in the stomach by stomach acids and enzymatic action, however, the overall bioavailability and/or therapeutic efficacy of a particular substance, including micro-organisms, can be impacted or greatly reduced by such stomach acids and enzymatic action, depending on the particular substance being orally ingested. Live and active probiotic cultures are one such example. Probiotics are living micro-organisms that naturally reside in the human intestine, and which scientific research has established are vital to a properly functioning immune system, and to our overall physical health and well-being. Through a variety of factors such as disease or the use of antibiotics, the normal balance of “good” versus “bad” bacteria in the intestine can be damaged or seriously impaired, and can even be fatal if left untreated. These imbalances in the microbiome of the intestine have been shown to have other important and vital effects on our health. Many of these imbalances can be, and have been, successfully addressed and treated through the use of live and active probiotic cultures.

In this regard, the US Food and Drug Administration (FDA) and World Health Organization in 2002 recommended that “the minimum viable numbers of each probiotic strain at the end of the shelf-life” be reported on labeling. However, but most companies that give a number report the viable cell count at the date of manufacture, a number probably much higher than that which exists at the moment of consumption. Because of variability in storage conditions and amount of time that has elapsed before consuming probiotics, it is difficult to tell exactly how much live and active culture remains at the time of consumption. Due to these ambiguities, the European Commission placed a ban on putting the word “probiotic” on the packaging of such products because such labeling can be misleading.

As a result, most probiotics are either not alive when they are taken orally (and are therefore completely ineffective for recolonizing the gut with “good” or healthy bacteria), or if alive when taken, are often destroyed in the stomach by stomach acids and enzymatic action, leaving a relatively small amount, if any, of the probiotics that actually make it to the small intestine alive and intact, where they are then able to recolonize the gut with the “good” bacteria, or address certain flora deficiencies as needed. As a result of this problem, patients suffering from a Clostridium difficile infection (CDI), for example, must often resort to fecal microbiota transplants (FMT), in order to restore the colonic microflora by introducing healthy bacterial flora directly into the large intestine.

An easier and more cost-effective method of delivering sufficient numbers of live and active probiotic cultures into the small intestine by oral delivery would clearly be a substantial improvement over fecal transplants. In addition, oral delivery of sufficient numbers of live and active probiotics to treat lesser conditions and for the general maintenance of the gut microbiome, with its attendant health benefits, would constitute a significant improvement over current oral delivery methods, which can be inefficient and largely ineffective.

Another problem with many drugs, medicines and other bioactive and therapeutic substances currently administered orally is that oral ingestion of these substances can cause severe stomach upset, nausea, and/or vomiting. These adverse effects are well-know and well-documented, and often appear on the warning label for the medicine as potential side effects. Common aspirin, for example, many prescription pain medications, and many chemotherapy drugs and other medications can, and often do, cause severe stomach upset, nausea, and/or vomiting when taken orally. Millions of people around the world suffer daily from these negative and unpleasant side effects when taking various medications, and so a viable, cost-effective solution is needed and would be a welcome relief to millions of people.

As one example, popular and widely used prescription pain relief medications, many of which are comprised of opioid derivatives such as oxycodone, hydrocodone, codeine, morphine, fentanyl and others, cause stomach upset, nausea, and/or vomiting. These opioid-derived pain medications interact with opioid receptors in the brain and nervous system in order to relieve pain. There were about 300 million pain medication prescriptions written in 2016.

There are two major problems with prescription opioid-based and other pain medications. Number one, they cause stomach upset, nausea, and/or vomiting in a large number of people who take them as previously mentioned. Number two, they are routinely crushed into a powder by drug dealers, and sold to addicts and others who inhale, snort or smoke the powder, or liquify it and inject it directly into their veins or arteries. As a result of this fact, the U.S. is in the midst of a massive opioid epidemic that has been widely reported on and discussed in the media. It is estimated that in 2015 more than 33,000 people died from overdoses of prescription pain medications in the U.S. Annually, opioids kill more people than car accidents and guns, and are now the leading cause of accidental deaths in the U.S.

It would be highly desirable and beneficial to be able to (i) administer pain and other medications orally without encountering any of the negative side effects commonly associated with taking such medications orally (upset stomach, nausea, and vomiting), and (ii) increase the bioavailability and thereby the efficiency of the pain medication, thereby reducing the amount of the pain medication (or dose) required, and (iii) create an oral delivery method which prevents opioid-based pain medications from being concentrated or crushed into a powdered form for use by drug addicts and black market sellers, or makes it prohibitively difficult or expensive to do so.

In the case of the administering medicines and other bioactive and therapeutic substances orally to animals, including pets and livestock, there is the additional problem that many medicines and other bioactive substances do not taste good to the animal, and therefore the animal will refuse to take or eat the medicine or other substance, or will spit out all or a portion of the medicine or other bioactive substance, making it difficult to administer these therapeutic substances to animals. This also results in not knowing exactly how much medicine the animal has taken or ingested, and therefore creates uncertainty as to how effective the unknown dose taken will actually be. The oral administration of medicines and other bioactive substances to animals can also create unnecessary anxiety and trust issues between the animal and the person administering the medicine or other therapeutic substance, and bites and other injuries to persons administering such oral medications and other substances to animals have frequently occurred. This process can also result in the substantial additional expense of having to hire and use a veterinarian or other trained professional to successfully administer the medicine to the animal by injection or other means. Given the widespread nature of these problems, a viable and cost-effective solution would be beneficial and welcome.

Certain vitamins and other dietary supplements are essential to our health and well-being, and evidence-based clinical research supports their importance and wide-ranging health benefits. Among them are Vitamin D, Coenzyme Q10, and Omega-3 fatty acids (EPA/DHA). Omega-3 fatty acids are often sold in the form of fish or Krill oil, or are sold as supplements in a variety of forms. As a result of the established health benefits of these and other vitamins and dietary supplements, they are often recommended or prescribed by physicians. Evidence-based clinical research also strongly suggests these and other dietary supplements should be incorporated into many diets to ensure that sufficient amounts of these critical substances are available for our overall health and well-being.

With respect to omega-3 fatty acids, for example, research has shown that cultures that routinely eat foods with high levels of omega-3 fatty acids demonstrate a variety of health benefits, such as lower levels of depression. Omega-3 fatty acids may also aid in treating the depressive symptoms of bipolar disorder, and may be important for visual and neurological development in infants. When ingested in relatively high doses, it may lower inflammation, which may be important in treating asthma. Other research suggests omega-3 fatty acids may be useful in ameliorating and/or reducing symptoms associated with ADHD in some children, while at the same time enhancing their mental skills. Omega-3 fatty acids may also prove to be useful in the treatment or slowing the progression of Alzheimer's disease and dementia.

With respect to Vitamin D, research has shown it can be important in reducing inflammation (by acting on C-Reactive Protein). It is also thought to aid in reducing pain as well as the stress on joints. Vitamin D has also been implicated as a possible source of reducing rheumatoid arthritis, obesity, certain cancers, various heart diseases, and the effects of radiation, while enhancing individuals' mental capacity, the immune system, bone growth, and the proper production of insulin. Although vitamin D can be procured by exposure to sunlight and other ways, vitamin D can also be attained by oral administration in a supplement form.

With respect to Coenzyme Q₁₀, it is a substance that helps convert food into energy, is found in almost every cell in the body and it is a powerful antioxidant. It is also critical in fulfilling the energy requirements of different organs such as the liver, heart and kidney. It is soluble in oil and present in most eukaryotic cells such as mitochondria. CoQ₁₀ is involved in the electron transport chain and participates in aerobic cellular respiration which generates energy. Over ninety percent of the human body's energy is generated this way. CoQ₁₀ is widely used in numerous applications as an antioxidant. There is also increasing use of CoQ₁₀ in medical applications like heart disease, eye care, cancer treatment, obesity and Huntington's disease.

These and other oil-based dietary supplements, however, face the industry-wide problem of oxidation, which results in the formation of toxic peroxides and other undesirable substances. This oxidation results in degradation of the substance, spoliation, and often a foul and offensive smelling odor and bad breath, all of which can be a strong disincentive for purchasing or taking these supplements again. As a result, dietary supplements such as omega-3 fatty acids, CoQ₁₀ and vitamin D are hampered by oxidation in storage, as well as by the intrinsic properties of the digestive tract, especially the pH differential along the digestive tract. The variable pH from the stomach to the intestine impacts the stability of the substance, and thereby the bioavailability of fat and peptide-based dietary supplements and other bioactive substances. Thus, the bioavailability of these and other dietary supplements are hampered by oxidation in storage, and by the digestive process in the stomach when taken orally.

As a result of these and other problems associated with the oral administration of various bioactive substances, it would be highly desirable and beneficial to have a method of orally delivering these substances to humans and animals, which increases bioavailability and, at the same time, eliminates many of the problems associated with the current oral delivery of these bioactive substances, some of which were discussed above. The invention described in this application was developed to address and solve these and other problems associated with the delivery of such bioactive substances to humans and animals.

### BRIEF SUMMARY OF THE INVENTION

A novel chemically modified alginate hydrogel has been developed which combines an aromatic compound with a carbohydrate, where the aromatic compound is one or more amines combined with an alginate. The chemical structure of alginate is modified using different amines and different methods, including: (i) covalently bonding aminoethyl benzoic acid to the alginate backbone, and (ii) oxidizing the vicinal diol in the alginate chain to an aldehyde before coupling to aminoethyl benzoic acid. Alternatively, the combined aromatic compound and carbohydrate can be a dopamine combined with the alginate. The chemically modified alginate and methods used can be utilized to encapsulate a variety of bioactive substances for oral delivery in humans and animals, including, but not limited to: (i) medicines, drugs, enzymes, proteins, hormones, and vaccines, (ii) vitamins, minerals, micronutrients and other dietary supplements, (iii) probiotics and other micro-organisms, (iv) cells, cell parts, and/or other biological materials, and/or (v) many other bioactive substances.

As used herein, the term “bioactive substance” means a substance used by or having any biological effect on a living organism, and includes, but is not limited to, prescription and non-prescription medications and drugs, chemicals, chemical compounds, molecules, enzymes, proteins, hormones, vaccines, vitamins, minerals, micronutrients and other dietary supplements, probiotics and other micro-organisms, cells, cell parts (including DNA and RNA), and other biological materials, as well as other bioactive compounds and substances.

Current oral delivery methods suffer from a number of significant drawbacks and limitations, depending on the substance being delivered. Chief among these is the fact that many substances taken orally are attacked, degraded and/or destroyed in the stomach by stomach acids and/or enzymatic action. Even if a substance is not completely destroyed in the stomach by stomach acids and enzymatic action, the overall bioavailability and/or therapeutic efficacy of a particular bioactive substance can be impacted or greatly reduced by such stomach acids and enzymatic action. Another problem with many drugs, medicines and other therapeutic substances taken orally is that oral ingestion of these substances can cause severe stomach upset, nausea, and/or vomiting.

The invention relates to a method of protecting the medicine or other bioactive substance from attack by acids and enzymatic action in the stomach by encapsulating the medicine or other bioactive substance in the modified alginate hydrogel. When the encapsulated medicine or other bioactive substance reaches the small intestine, it is released into the small intestine by diffusion due to the pH differential, or as the microcapsule falls apart, thereby increasing the overall bioavailability and effectiveness of the medicine or other bioactive substance. This method of encapsulation and oral delivery also eliminates certain problems and adverse side effects often associated with the oral delivery of various medicines and other bioactive substances in humans and animals. Accordingly, this novel modified alginate hydrogel, its methods of preparation, and its uses, whereby medicines and other bioactive substances are encapsulated for oral delivery to humans and animals, can provide a wide variety of health benefits, while eliminating certain problems and adverse side effects often associated with the oral delivery of these medicines and other bioactive substances.

Among other things, various compositions and combinations which comprise the invention may also be micro-encapsulated in the modified alginate and produced in a size suitable for injection, either by itself, or in combination with liposomes, micelles, and/or nanospheres for, among other things, targeted delivery to a specific site or group of cells in humans or animals, such as a tumor site.

In one embodiment, the combined aromatic compound and carbohydrate is one or more amines combined with the alginate. In another embodiment, the combined aromatic compound and carbohydrate is dopamine combined with the alginate. In one embodiment, for example, the combined aromatic and carbohydrate is 4(2-ethylamino)benzoic acid alginate. In another embodiment, the combined aromatic compound and carbohydrate is dopamine alginate.

In one embodiment, the aromatic substituent is an amine substituent, including, but not limited to, a 4(2-ethylamino)benzoic acid derivative, a 4(2-ethylamino)phenolic derivative, a 4(2-ethylamino)anilinic derivative, or a para (2-ethylamino)toluenic (i.e., (2-ethylamino)4-methylbenzene) derivative, and/or mixtures thereof.

In one embodiment, the aromatic substituent is a dopamine substituent, including, but not limited to, a 4(2-ethylamino)phenolic substituent, a 4(2-ethylamino)benzoic acid substituent, a 4(2-ethylamino)anilinic substituent, a 4(2-ethylamino)toluenic substituent, and/or mixtures thereof.

In an embodiment, the present invention relates to alginate compounds and methods of preparing said alginate compounds for encapsulating the bioactive substance. In one variation, the alginate is a dopamine-modified alginate (DMA) using 2 different preparations. These two different preparations can be characterized and quantified by a ¹H-NMR methodology that was developed for quantifying dopamine incorporation into the alginate backbone.

By preparing the compounds of the present invention, it has been found that the alginate encapsulated compounds are protected at pH levels that are found in the stomach (pH of about 1-3) but the alginate encapsulated compounds are able to be made available at the pH levels that are found in the intestines (more basic pH levels of about 7-9) as the alkaline environment allows the alginate compound to be broken down and thus release of the encapsulated compound is achieved. The alginate encapsulated compounds are protected at acidic pH levels by the alginate coupled to the aromatic amine compound.

### DETAILED DESCRIPTION OF THE INVENTION

A novel chemically modified alginate hydrogel has been developed which combines an aromatic compound with a carbohydrate, where the aromatic compound is one or more amines combined with an alginate. The chemical structure of alginate is modified using different amines and different methods, including: (1) covalently bonding aminoethyl benzoic acid to the alginate backbone, and (2) oxidizing the vicinal diol in the alginate chain to an aldehyde before coupling to aminoethyl benzoic acid. Alternatively, the combined aromatic compound and carbohydrate can be a dopamine combined with the alginate. The chemically modified alginate and methods used can be utilized to encapsulate a variety of bioactive substances for oral delivery in humans and animals, including, but not limited to: (i) drugs, medicines, enzymes, proteins, hormones, and vaccines, (ii) vitamins, minerals, micronutrients and/or other dietary supplements, (iii) probiotics and/or other microorganisms, (iv) cells, cell parts, and/or other biological materials, and/or (v) other bioactive substances.

Current oral delivery methods suffer from a number of significant drawbacks and limitations, depending on the substance being orally ingested. Chief among these is the fact that many substances taken orally are attacked, degraded and/or destroyed in the stomach by stomach acids and/or enzymatic action. Even if a substance is not completely destroyed in the stomach by stomach acids and enzymatic action, the overall bioavailability and/or therapeutic efficacy of a particular bioactive substance can be impacted or greatly reduced by such stomach acids and enzymatic action, depending on the substance being taken orally. Another problem with many drugs, medicines and other therapeutic substances administered orally is that oral ingestion of these substances can cause severe stomach upset, nausea, and/or vomiting.

The invention relates to a method of protecting the medicine or other bioactive substance from attack by acids and enzymatic action in the stomach by encapsulating the medicine or other bioactive substance in the modified alginate hydrogel. When the encapsulated medicine or other bioactive substance reaches the small intestine, it is released into the small intestine by diffusion due to the pH differential, or as the microcapsule falls apart, thereby increasing the overall bioavailability and effectiveness of the medicine or other bioactive substance. This method of encapsulation and oral delivery also eliminates certain problems and adverse side effects often associated with the oral delivery of various medicines and other bioactive substances in humans and animals. Accordingly, this novel modified alginate hydrogel, its methods of preparation, and its use to encapsulate medicines and other bioactive substances for oral delivery to humans and animals, can provide humans and animals with a wide variety of health benefits, while eliminating certain problems and adverse side effects often associated with the oral delivery of these medicines and other bioactive substances.

Among other things, the various compositions and combinations which comprise the invention may also be encapsulated and produced in a micro-size suitable for injection, either by itself, or in combination with liposomes, micelles, and/or nanospheres, for targeted delivery to a specific site or group of cells in humans or animals, such as a tumor site.

### Modifying the Alginate

In an embodiment, the combined aromatic compound and carbohydrate is one or more amines combined with the alginate. In another embodiment, the combined aromatic compound and carbohydrate is a dopamine combined with the alginate. In an embodiment, the combined aromatic compound and carbohydrate is 4(2-ethylamino)benzoic acid alginate. In another embodiment, the combined aromatic compound and carbohydrate is a dopamine alginate.

In an embodiment, a 4(2-ethylamino)benzoic acid derivative, a 4(2-ethylamino)phenolic derivative, or a 4(2-ethylamino)anilinic derivative, and/or a para (2-ethylamino)toluenic (i.e., (2-ethylamino)4-methylbenzene) derivative is used to create the modified alginate. In an embodiment, the methodology used involves using N-Hydroxysuccinimide (NHS) optionally in conjunction with 1-Ethyl-3-(3-dimethylaminopropyl)carbodiimide (EDC) to activate the carboxylic acid (or carboxylate) on the alginate allowing the primary amine on the aromatic substituent to react with the carboxylate to generate the amide.

In an embodiment, the chemical structure of naturally occurring alginate is also covalently and noncovalently modified, by adding catechol functional groups to the alginate backbone with the goal of improving alginate's adhesive properties and its rigidity thereby improving its performance as a wound healing aid or surgical adhesive.

In an embodiment, generically one can react a carbohydrate with an aromatic compound (wherein both have reactive functionalities) to generate a polymer that contains both a carbohydrate portion and an aromatic portion. For example, in an embodiment, the reaction may proceed as indicated below in Scheme I wherein X is a counterion that allows the carboxylate to make a salt. R₁ may be any of a plurality of substituents such as OH, NH₂, COOH, NO₂, CN, Br, Cl, F, C₂₋₆alkylhalides, CH₃, C₍₂₋₆₎alkyl, SO₃H, or COCH₃. m is 0 to 3, n is 1 to 1,000,000, and o is 1 to 3. It should be noted that R₁ can be a mixture of substituents. That is, for example, if o is 2, R₁ can be two hydroxyls, or alternatively, one of the R₁'s can be hydroxyl and the other R₁ can be a halide (or any other substituent identified herein).

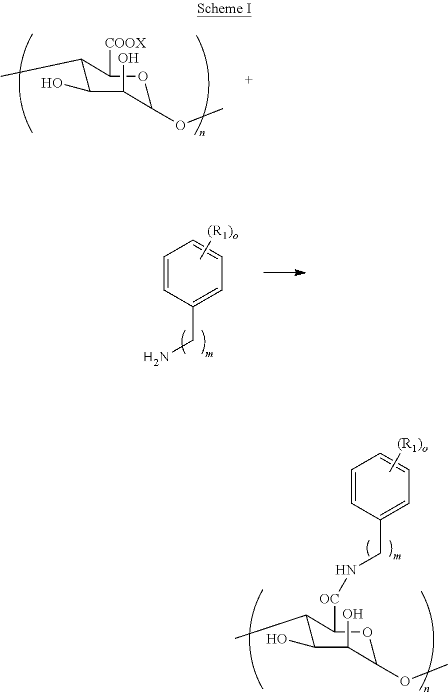

The reaction as shown in the scheme immediately above can be done using alginates (as the carbohydrate). Alginic acid is a combination of β-D-mannuronic and α-L-guluronic acids attached with 1→4 linkages. Thus, although scheme I is shown with only one type of carbohydrate, it should be understood that the respective sugars in the carbohydrate may be different. Similar to using alginic acid, it should be understood that other types of carbohydrates may be used.

In an embodiment, the carbohydrate combined with an aromatic are such that the carbohydrate is alginic acid, X is sodium, m is 2, n is between about 500,000 and 1,000,000, o is 2, and the two R₁ are both hydroxyls. In one embodiment, the hydroxyls are positioned meta and para to the ethylamine (that is present on the phenyl group). In another embodiment, the carbohydrate is alginic acid, X is sodium, m is 2, n is between about 500,000 and 1,000,000, o is 1, and R₁ is amino or a carboxylate salt (or carboxylic acid).

In an embodiment, the aromatic substituent that reacts with alginic acid (or other carbohydrate substituent) can alternatively be 4-aminomethyl benzene sulfonamide or 4-aminoethyl benzene sulfonamide. Similarly to the reaction as shown in scheme 1, if 4-aminomethyl benzene sulfonamide or 4-aminoethyl benzene sulfonamide is used. n may be between about 500,000 and 1,000,000, the 4-aminomethyl benzene sulfonamide or 4-aminoethyl benzene sulfonamide may contain additional substituents off the benzene ring, and the substituents in one embodiment may be amino or a carboxylate salt (or carboxylic acid).

Generally, the reaction is performed in a buffer such as PBS (Phosphate Buffered Saline), but other buffers are contemplated that can be used (as long as they don't adversely affect the reaction). Additionally, and/or alternatively, the reaction may take place in a nitrile such as acetonitrile.

Although the reaction above in scheme I is shown with varying linker group sizes (m can be 0 to 3), and different functional groups on the aromatic ring, maximal microencapsulation may occur when the linker group is an ethylene. It should be recognized that in order to get the desired reaction to proceed, protective groups may be utilized to avoid having a plurality of different reaction processes occurring. These protective groups and their chemistry can be found in, for example, Greene's Protective Groups in Organic Synthesis, Fourth Edition, 2007, John Wiley & Sons, Inc., which is hereby incorporated by reference in its entirety.

In an embodiment, the present invention relates to alginate compounds and methods of preparing said alginate compounds for encapsulating the bioactive substance to be delivered orally or by other means. In one variation, the alginate is an amine-modified alginate. In another variation, the alginate is a dopamine-modified alginate (DMA) using 2 different preparations. These two different preparations can be characterized and quantified by a 1H-NMR methodology developed for quantifying dopamine incorporation into the alginate backbone.

Dopamine (i.e., (4-(2-aminoethyl) benzene, 1,2-diol)), for example, has three substituents, including a ethylene linker with a reactive amino group that allows it to be ideally linked to alginate (or alginic acid). Without being bound by theory, the linker group is of a sufficient length to allow ideal microencapsulation¹ of medicines, drugs, proteins, hormones, vaccines, vitamins, minerals, micronutrients and other dietary supplements, biological materials, probiotics and other micro-organisms, and other bioactive compounds and substances. The modified alginate has both a polar group (the carbohydrate portion) and a hydrophobic portion (the aromatic benzene) ring that allows ideal microencapsulation. The modified alginate compounds of the present invention are ideal for protecting the bioactive substance from attack and degradation by acids and enzymatic action in the stomach, thereby enhancing the bioavailability and effectiveness of these bioactive substances in the place where they are most useful and beneficial (i.e., in the intestines).

By preparing the compounds of the present invention, it has been found that the alginate encapsulated compounds and other substances are protected at pH levels that are found in the stomach (pH of about 1-3) but the alginate encapsulated compounds are able to be made available at the pH levels that are found in the intestines (more basic pH levels of about 7-9) as the alkaline environment allows the alginate compound to be broken down and thus access to the encapsulated compound is achieved. The alginate encapsulated compounds and substances are protected at acidic pH levels by the dopamine alginate.

In an embodiment, the lability of the alginate encapsulating material is such that the alginate encapsulating material is able to withstand the pH of saliva (generally a pH of about 6.5-7.4) for a sufficient amount of time that the alginate encapsulating compounds are able to reach the stomach in a still encapsulated state. Then, upon prolonged exposure to the pH of the small intestine, the encapsulated compounds become bioavailable for their intended benefits. In an embodiment, the alginate compound can be made by the scheme shown in scheme 11.

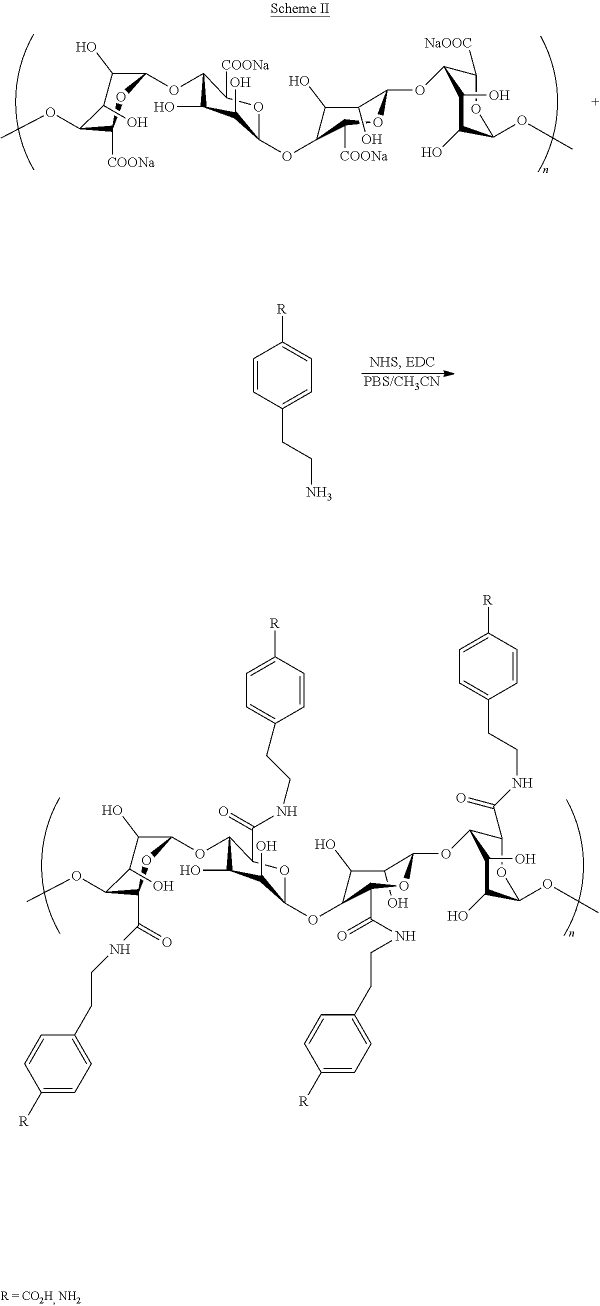

To accomplish alginate modification, the present invention relates to simultaneously pursuing two approaches: 1) formation of amide bonds to existing carboxylic acid groups on the alginate backbone and 2) synthesizing small molecules containing catechol functional groups which can be used as modular additives to other available alginate systems. Some of these small molecules are covalently linked to alginate and some interact through noncovalent interactions such as hydrogen bonding. The results of these approaches indicate that both stiffness and adhesiveness of alginate can be improved by up to a factor of three with small molecule additives. It has been unexpectedly found that the preparation and addition of the modular small molecules to alginate using the first approach indicated above only takes a matter of hours whereas the more classical second approach of forming amide bonds to the polysaccharide first and then using that modified polysaccharide takes days.

In an embodiment, using the methods of the present invention allows one to incorporate greater amounts of dopamine (or other aromatic substituents) into the alginate than has been previously reported. Using the methods of the present invention allows between about 5% to about 15% incorporation of dopamine into the alginate. Thus, in an embodiment the present invention relates to the incorporation of dopamine into alginate at a level of between about 5-15% by weight, alternatively between about 5-12%, alternatively between about 5-10%, alternatively between about 10-15%, or alternatively between about 5-8%.

In an embodiment, the present invention relates to customized cross-linked alginate-amine, or other aromatic coatings, for encapsulating medicines, drugs, enzymes, proteins, hormones, vaccines, vitamins, minerals, micronutrients and other dietary supplements, biological materials, probiotics and other micro-organisms, and other bioactive compounds and substances, and methods of applying the same. The amine and dopamine alginates are produced using a reaction scheme that does not require elevated temperatures. The techniques disclosed herein enable application of molecular monolayers of the alginate-amine or alginate-dopamine coatings on the bioactive substance, and on probiotics and other micro-organisms as well. These monolayer coatings can be on the order of nanometers in thickness.

It has been unexpectedly discovered that the coatings disclosed herein enable application of a coating that allows these various medicines, drugs, enzymes, proteins, hormones, vaccines, vitamins, minerals, micronutrients and other dietary supplements, biological materials, probiotics and other micro-organisms, and other bioactive substances to be used by a host organism, such as humans and animals, without causing the premature rupture of the encapsulated material, for example in the acidic environment of the stomach.

In one embodiment, the invention is drawn to a biocompatible capsule that includes a biological material and a covalently stabilized coating encapsulating the medicine or other bioactive substance.

The present invention offers unexpectedly superior properties and results because the modification of alginate by attachment of amine or dopamine molecules results in enhanced adhesiveness of the alginate polysaccharide.

In an embodiment, the present invention also relates to the transport of solutes within alginate hydrogels. Transport of solutes is important for success in both protein and cell delivery systems. The transport within this hydrogel system is largely driven by diffusion, and diffusion varies as a function of alginate composition and concentration. The rate of diffusion is dependent on the G fractions of alginate, with the diffusion coefficient increasing at lower G fractions. This is attributed to the flexibility of the polymer backbone, meaning that higher G fractions result in higher crosslinking, less swelling and hence a greater barrier to diffusion. The measurements of simple physical parameters, such as volume fraction and size, can be used to predict solute transport in alginate hydrogels. These parameters can be controlled based on the alginate concentration and composition for sustained release of small amounts of substances encapsulated in alginate.

However, in situations where the release of readily effective therapeutic levels is desired, the present invention offers the benefit of modifying the alginate delivery vehicle to release the encapsulated products based on prompt degradation of the alginate hydrogel. The present invention is able to achieve this immediate release and enhance the bioavailability of therapeutic molecules encapsulated in alginate hydrogel by modifying the alginate polymer to degrade based on sensitivity to the basic pH of the small intestine where absorption into the systemic circulation also takes place.

In an embodiment, and in the case of alginate, covalent modifications of the polysaccharide have been performed to alter the acid-base stability of alginate pellets used for drug delivery. The results show that amine and dopamine modified alginates are stable in acid environments similar to what one would find in the stomach and that these pellets disintegrate readily in a basic environment similar to what one would find in the small intestine. In a variation, the present invention aims to synthesize alginates with varying degrees of amide modification and to prepare alginates of additional amides in order to optimize properties for acid and base sensitive drug delivery applications. In a variation, the modified alginates will be formulated into microparticles and optimize tested for their acid-base stability (amide-modified). In an embodiment, the modified carbohydrate formulations/microparticles may also be used for wound healing aid or used as surgical adhesives.

Prior to the present invention, to the inventors' knowledge, there was no report of alginate modification by use of amines, or alginate modification such that its hydrogel would readily degrade in response to a basic pH sensitivity to release encapsulated products in the small intestine. Thus, a unique phenomenon that would enhance the bioavailability of therapeutic agents, such as probiotics and other bioactive substances, is contemplated, and therefore within the scope of the present invention.

### Encapsulating Various Bioactive Substances

In an embodiment, the present invention relates to protecting a multitude of bioactive substances from the destructive effects of acids and enzymatic action in the stomach, and thereby enhancing the bioavailability and effectiveness of these various bioactive substances by encapsulating them in one or more embodiments of the modified alginate.

In one embodiment, the present invention relates to encapsulating proteins, hormones, and other bioactive substances in the modified alginate, which protects these bioactive substances from attack and degradation by acids and enzymatic action in the stomach, which bioactive substances are then released in the intestines as the modified alginate micro-capsule falls apart and/or diffuses its contents into the intestines. One such example is encapsulating insulin in the modified alginate in a bioavailable form for oral delivery to protect it from destruction by acids and enzymatic action in the stomach, so that the insulin can be delivered to the small intestine intact, where it can be released and absorbed into the bloodstream for use by humans or animals in appropriate amounts.

In an embodiment, the present invention relates to encapsulating drugs, medicines, and other bioactive substances in the modified alginate, such as non-prescription pain medications, so that these substances do not cause stomach upset, nausea, and/or vomiting when taken orally. One example is encapsulating aspirin (acetylsalicylic acid) for oral delivery, thereby preventing the release of the acetylsalicylic acid in the stomach, where it often causes stomach upset, nausea, vomiting, and even ulcers (if taken regularly to reduce pain or inflammation). Instead, the encapsulated aspirin is released in the small intestine as the modified alginate micro-capsule falls apart and/or diffuses its contents into the intestines, where it is absorbed into the bloodstream to reduce fever, relieve pain, swelling, and inflammation, from conditions such as muscle aches, toothaches, common cold, flu, headaches, and arthritis; prevent blood clots and lower the risk of heart attack, clot-related strokes and other blood flow problems in patients who have cardiovascular disease, or who have already had a heart attack or stroke; and to treat a variety of other conditions in humans and animals.

In an embodiment, the present invention relates to encapsulating drugs, medicines and other bioactive substances in the modified alginate, such as prescription pain medications, so that these substances do not cause stomach upset, nausea, and/or vomiting when taken orally. An example is encapsulating opioid-based pain medications such as oxycodone, hydrocodone, codeine, morphine, fentanyl and others. These medications often cause stomach upset, nausea, and/or vomiting when taken orally. When these pain medications are encapsulated in the modified alginate for oral delivery, the modified alginate prevents the release of the pain medication in the stomach, where it would normally cause stomach upset, nausea, or vomiting. Instead, the encapsulated pain medication is not released until it reaches the small intestine, where the modified alginate micro-capsule falls apart and/or diffuses its contents into the intestines, where it is absorbed into the bloodstream to reduce moderate to severe pain from a variety of injuries, diseases and other serious or life threatening conditions.

In an embodiment, the present invention relates to encapsulating opioid-based and other potentially addictive pain medications in the modified alginate for oral delivery, so that the prescription pain medications cannot easily or readily be separated from the modified alginate, turned into a powder, and sold by drug dealers to drug addicts, who inhale, snort or smoke the powder, or liquify it and inject it directly into their veins or arteries.

In an embodiment, the present invention relates to encapsulating medicines and other bioactive and therapeutic substances in the modified alginate to increase bioavailability, protect the substance from attack, degradation or destruction by acids and enzymatic action in the stomach, and make it largely tasteless, odorless and undetectable for oral delivery to animals, including pets and livestock. The largely tasteless, odorless, and undetectable micro-capsules containing the medicine or other bioactive substance can then be combined with pet food, animal feed, and other foodstuffs the animal finds appealing, so that the animal will readily eat the micro-encapsulated medicine or other bioactive substance, and not reject it or spit it out, as is often the case with any food or other substance that does not smell good or taste good to the animal, which also makes it difficult to administer these therapeutic substances to pets, livestock, and other animals. This novel method of encapsulation of medicines and other bioactive substances for oral delivery to animals will also (i) increase the bioavailability of the encapsulated medicine or other bioactive substance being ingested, (ii) make it easier to gauge the amount of the bioactive substance that is actually ingested by the animal, (ii) reduce the amount of medicine or other bioactive substance required (dose), since greater bioavailability (i.e., greater efficiency and effectiveness of the medicine) often results in a lower dose being required, (iii) eliminate unnecessary anxiety and trust issues between the animal and the person administering the medicine or other therapeutic substance, (iv) eliminate the risk of injuries to persons administering the medicines and other bioactive substances to the animal, and (v) eliminate the cost and expense of having to utilize a veterinarian or other trained professional to administer the medicine or other bioactive substance to the animal.

In an embodiment, the present invention relates to encapsulating live and active probiotics, gut flora, and other “good” or “healthy” micro-organisms in the modified alginate to protect them from attack, degradation, and/or destruction by acids and enzymatic action in the stomach. The micro-organisms are then released in the intestines alive and intact, where their health and other therapeutic benefits can be fully realized. One example is encapsulating Lactobacillus Casei NCDC 298 in the modified alginate. Encapsulating probiotics both lengthens their shelf-life and shields them from attack, degradation, and/or destruction by acids and enzymatic action in the stomach after they are orally ingested. When the encapsulated probiotics reach the small intestine, they are released as the modified alginate falls apart. The probiotics are then able to recolonize the gut with their “good” bacteria, so that the many health and other therapeutic benefits of the probiotics can be fully utilized and realized. Many other types and strains of probiotics can be encapsulated in the modified alginate for oral delivery to humans and animals.

The maintenance of live bacterial cells until they are able to reach the intestines is one of the key requirements for obtaining health benefits from probiotics. Therefore, in an embodiment, the present invention relates to providing probiotic living cells with a physical barrier against adverse environmental conditions until delivery to the intestines has been accomplished. In a variation, the proper conditions are a basic pH. In an embodiment, the present invention relates to a composition that includes an encapsulated probiotic that has a plurality of health benefits.

Because probiotics are biological entities, delivery of sufficient doses is constantly challenged by inherent factors that might limit their biological activity, including the conditions of growth, processing, preservation, and storage. Specifically, loss of probiotic viability occurs at many distinct stages, including freeze-drying of cells during initial manufacturing, during their preparation (high temperature and high pressure), transportation and storage (temperature fluctuations), and after consumption or in gastrointestinal (GI) track (low pH and bile salts). One of the determined factors for probiotics to have beneficial effects is to maintain the high concentration of viable cells for individuals to uptake. Although commercial probiotic products are available, many of them lose their viability during the manufacturing process, transport, storage.

In an embodiment, the present invention relates to a composition which contains a probiotic. In one embodiment, the compositions of the present invention may be good for those that have cardiovascular issues. The composition of the present invention may be useful at improving the immune health of individuals that consume the composition. In one embodiment, the composition of the present invention may comprise both a prebiotic, which optimizes the conditions for any composition, that also contains probiotics.

In an alternate embodiment, the composition of the present invention may contain one or more probiotic cultures that may include, for example, various species of the genera Bifidobacterium, Lactobacillus, and propionibacteria such as: Bifidobacterium animalis lactis; Bifidobocterium bifidum; Bifidobacterium breve; Bifidobacterium infantis; Bifidobacterium longum; Lactobacillus acidophilus; Lactobacillus casei; Lactobacillus plantarum; Lactobacillus reuteri; Lactobacillus rhamnosus; Lactobacillus spoogenes and the like. A species of yeast Saccharomyces boulardii, may also be used as a probiotic. In an embodiment, the probiotic cultures include Bifidobacterium lactis BI-04, Bafidobacterium lactis BB-12 (CHN), and L. reuteri (SD 55730-Biogaia).

In an embodiment, the present invention relates to encapsulating vitamins, dietary supplements, and other bioactive substances in the modified alginate for oral delivery to humans and animals, since certain vitamins, dietary supplements, and other bioactive substances suffer from issues of bioavailability, as well as issues of oxidation and the build-up of toxic peroxides and other substances, among other things. These include Omega-3 fatty acids (EPA/DHA), CoQ10, and vitamin D, which current research strongly suggests are important to our overall health and well-being, and should be administered and used to supplement the diets of large numbers of people around the world. Among other things, the bioavailability of these vitamins, dietary supplements, and other bioactive substances, and their ability to be stored for any length of time, are hampered by oxidation and the build-up of toxic peroxides and other substances. They can also be impacted by the intrinsic properties of the digestive tract, especially the differential pH along the tract. The variable pH from the stomach to the intestine impacts the stability, and thereby the bioavailability, of fat and peptide-based dietary supplements and other pharmaceuticals. Some vitamins and dietary supplements, such as vitamin C, vitamin B3, vitamin A and vitamin D, can also cause stomach upset, nausea, and vomiting when taken orally. When these vitamins, dietary supplements and other bioactive substances are encapsulated in the modified alginate for oral delivery, encapsulation (i) protects the encapsulated substance from the destructive and toxic effects of oxidation while being stored, (ii) prevents the release of the encapsulated substance in the stomach, where it causes stomach upset, nausea, or vomiting, (iii) protects the encapsulated substance from attack, degradation, and destruction from stomach acids and enzymatic action, and (iv) prevents the encapsulated substance from being released until it reaches the small intestine, where the micro-capsule falls apart and/or diffuses its contents into the intestines, where the substance is absorbed into the bloodstream for its health and other benefits.

One such example is to increase the bioavailability of Vitamin D, which constitutes a largely unrecognized and serious public health problem. Chronic Vitamin D deficiency adversely affects adequate mineralization of bone and leads to rickets in children and osteomalcia or osteoporosis in adults. Low levels of 25-hydroxyvitamin D, the universal clinical parameter of vitamin D status, is associated with an increased risk of cancers, cardiovascular disease, and diabetes, among other diseases. Thus, the present invention relates to methods associated with being able to treat cancers, cardiovascular disease, diabetes, and other diseases. The present invention addresses issues that the dietary supplement and pharmaceutical industries have long considered necessary, but have been largely unavailable.

In an embodiment, the present invention relates to being able to prolong the bioavailability of medicaments/dietary supplements by combining a microencapsulated dietary supplement/medicament with the same or different non-microencapsulated medicament. The medicament/dietary supplement that is not microencapsulated will show bioavailability more rapidly (for example in the acidic stomach) whereas the microencapsulated dietary supplement/medicament will not be readily bioavailable until it passes through the acidic stomach. That is, it will be bioavailable once it passes to the more basic conditions of the intestines.

In an embodiment, the present invention relates to encapsulating cells, cell parts, tissues, and other biological materials in the modified alginate in a micro-size suitable for injection, either by itself, or in combination with liposomes, micelles, and/or nanospheres, for targeted delivery to a specific site or group of cells in humans or animals, such as a tumor site.

In embodiments of the present invention, the composition may be used to treat any of a number or maladies. For example, the functional aspects of the invention may act as an antioxidant, or treat digestive maladies and/or alternatively, treat cognitive disorders and or alternatively and/or additionally treat cardiovascular systems and diseases. The formulations of the instant invention may also be used to treat eczema. In an embodiment, a subject is a human in need of cancer treatment.

In an embodiment, the present invention relates to methods and compositions comprising the alginate hydrogel that can be used to micro-encapsulate a wide variety bioactive compounds and substances, which can then be administered orally to humans and animals in order to treat, and/or inhibit a wide variety of diseases, parasites, and other conditions in humans and animals, without significant degradation of the substance by stomach acids and enzymatic action, and without experiencing some of the negative side effects which often accompany certain medicines when taken orally.

In an embodiment, the present formulation may comprise a composition that contains one or more stilbenes sufficient to have desired antioxidant effects. Alternatively, in an embodiment, the one or more stilbenes present may have beneficial anti-inflammatory effects. In an alternate embodiment, the present invention may contain one or more stilbenes that are efficacious in reversing cognitive behavioral deficits. In an embodiment, the formulations of the present invention may be effective against Alzheimer's.

The composition of the present invention may additionally contain pharmaceutically acceptable salts, solvates, and prodrugs thereof, and may contain antiseptics, astringents, diluents, excipients, carriers, micelles, liposomes, or other substances necessary to increase the bioavailability or extend the lifetime of the compounds/probiotics present in the composition of the present invention. The present invention is not only directed to compositions but is also directed to formulations, supplements, sweeteners, medicaments, and other products and methods of using those products, formulations, supplements, and medicaments.

In an embodiment, the aromatic substituent comprises one or more amines, or mixtures thereof. In an embodiment, the aromatic substituent comprises one or more of a dopaminic substituent, a 4(2-ethylamino) phenolic substituent, a 4(2-ethylamino)benzoic acid substituent, a 4(2-ethylamino) anilinic substituent, a (2-ethylamino)4-methyl benzene (the toluene derivative) substituent or mixtures thereof. In one variation, the aromatic substituent comprises the dopaminic substituent.

In an embodiment, the modified alginate is stable under acidic conditions but is labile under basic conditions. In a variation, the modified alginate is stable at a pH of between about 1.5 to 3.5 but is labile when the pH increases to a level above 7.

In an embodiment, the amount of dopamine present in the modified alginate has between about 5% and 15% by weight dopamine.

In an embodiment, the present invention relates to a method of making proteins, micronutrients, dietary supplements and/or probiotics more bioavailable to an individual in need of said proteins, micronutrients, dietary supplements and/or probiotics by administering to said individual said proteins, micronutrients, dietary supplements and/or probiotics encapsulated in a modified alginate, the modified alginate being modified by the incorporation of covalently linked dopamine substituents, covalently linked 4(2-ethylamino) phenolic substituents, covalently linked 4(2-ethylamino)benzoic acid substituents, covalently linked 4(2-ethylamino) anilinic substituents, or covalently linked 4(2-ethylamino)toluenic substituents.

In a variation, the method is such that the amount of dopamine present in the modified alginate is between about 5% and 15% by weight dopamine. Alternatively, the amount of dopamine present in the modified alginate is between about 8% and 15% by weight dopamine.

In a variation of the method, the modified alginate is stable at a pH of around about 3 to 5 and labile at a pH above 7. In one variation, the microencapsulating alginate is stable for at least about 5 minutes at a pH above 7, or alternatively at least about 10 minutes, or alternatively at least about 15 minutes. In a variation, the microencapsulating alginate has a thickness such that the proteins, micronutrients, dietary supplements and/or probiotics can be exposed to saliva in the mouth and not be made bioavailable until the proteins, micronutrients, dietary supplements and/or probiotics reach the intestines of an individual that ingests the proteins, micronutrients, dietary supplements and/or probiotics.

In an embodiment, the present invention relates to a method of preparing an aromatic alginate, the method comprising reacting an alginate with an aromatic substituent, said aromatic substituent comprising one or more of a dopaminic substituent, a 4(2-ethylamino)phenolic substituent, a 4(2-ethylamino)benzoic acid substituent, a 4(2-ethylamino)anilinic substituent, a 4(2-ethylamino)toluenic substituent or mixtures thereof.

In a variation, the method of making the modified alginate incorporates a dopaminic substituent by reacting alginate with said dopaminic substituent, a 4(2-ethylamino)phenolic substituent, a 4(2-ethylamino)benzoic acid substituent, a 4(2-ethylamino)anilinic substituent, a 4(2-ethylamino)toluenic substituent in the presence of one or more of N-Hydroxysuccinimide (NHS) and 1-Ethyl-3-(3-dimethylaminopropyl)carbodiimide.

In one variation of the method, the solvent that is used in the method of making the modified alginate is a nitrile such as acetonitrile.

In an embodiment, the present invention relates to a method of delivering a dietary supplement or probiotic to an individual in need thereof, said method comprising administering to said individual a composition that comprises a modified carbohydrate, said modified carbohydrate comprising a modified alginate, that has been modified by an aromatic substitute, said aromatic substituent comprising one or more of a dopaminic substituent, a a 4(2-ethylamino)phenolic substituent, a 4(2-ethylamino)benzoic acid substituent, a 4(2-ethylamino)anilinic substituent, a 4(2-ethylamino)toluenic substituent or mixtures thereof.

In a variation, the present method uses proteins, micronutrients, dietary supplements and/or probiotics that are encapsulated by the modified carbohydrate.

In an embodiment, the modified carbohydrate is a modified alginate. In a variation, the modified alginate is modified by covalent addition of dopaminic substituents. In a variation, the modified alginate contains dopaminic substituents in an amount of about 5 to about 15% by weight dopamine. In an embodiment, the modified alginate is stable at a pH of about 3 to 5.

In an embodiment, the present invention relates to a method of treating an individual in need thereof by administering proteins, micronutrients, dietary supplements and/or probiotics to said individual, wherein the proteins, micronutrients, dietary supplements and/or probiotics are encapsulated in a modified alginate, the modified alginate being modified as described above. The method may include treating individuals for depression wherein the method uses as a dietary supplement fish oil that serves as a primary source for omega-3 fatty acids. The method may boost the ability to boost the effects of antidepressants, they also may aid in treating the depressive symptoms of bipolar disorder. The method may be also used for treating visual or neurological problems in infants or aiding visual and neurological development in infants. The method may allow ingestion of omega-3 fatty acids in relatively high doses that may lower inflammation, and may treat asthma.

In an embodiment, the invention relates to delivering omega-3 fatty acids to individuals, which are useful in ameliorating and/or reducing symptoms associated with ADHD in some children, while at the same time enhancing their mental skills. The invention also relates to the use of omega-3 fatty acids to treat or slow the progression of Alzheimer's disease and dementia.

In an embodiment, the invention relates to a method of using the modified alginate to deliver vitamin D. Thus, the method may be used to reduce inflammation (by acting on C-Reactive Protein). In a variation, the method of delivering vitamin D may aid individuals in reducing pain as well as the stress on joints. The method may also relate to the treatment of or reducing rheumatoid arthritis, obesity, certain cancers, various heart diseases, and the effects of radiation. Similarly, the method may be used to enhance individuals' mental capacity, the immune system, bone growth, and the proper production of insulin.

In an embodiment, the present invention relates to a method of administering insulin by using the methods and compositions as disclosed above. Thus, the method may be used as a means of keeping the blood sugar level from getting too elevated (hyperglycemia) or too low (hypoglycemia). In a variation, the method may be able to aid individuals who are unable to effectively produce the correct amount of insulin.

In an embodiment, the present invention also relates to a method of treating irritable bowel syndrome that allows the modified alginate to encapsulate a medicament that enhances the bioavailability of the medicament in the intestines where the medicament is most needed. Moreover, this would allow the delivery of medicaments that otherwise might be acid labile (that is, these medicaments are able to survive the acidic conditions of the stomach because they are encapsulated).

In an embodiment, Dopamine modified alginate using 2 different preparations were developed and a 1H NMR method for quantifying dopamine incorporation into the alginate backbone (Scheme 1). Alginate was prepared containing 4%, 8%, and 13% dopamine incorporation. Accordingly, in an embodiment, modified alginate was prepared that contained approximately 1 of every 25 carboxylates modified, 1/12 and 1/8, respectively.

In an embodiment, the 8% dopamine-modified alginate (DMA) was tested to make slabs encapsulating nanoparticles for oral drug delivery. After 2% weight % DMA was dissolved in Hank's Balanced Salt Solution (HBSS) without calcium, Omega-3 oil loaded silica nanoparticles were mixed with the DMA. The mixture was then crosslinked by adding CaCl2 and allowed to sit for about 15 minutes at room temperature until it formed a hydrogel slab. The hydrogel was cut in half to compare the degradation rate in different pH environments. One-half of the hydrogel was placed in a 1 N HCl (pH<1), and the other in a bath of Krebs Ringer Solution (pH 7.4) to mimic the highly acidic stomach, and the more neutral gut conditions, respectively. The hydrogel slabs under these two conditions were placed in an incubator at 37° C. and an inverted light microscope was used to compare the overall shape, transparency, and release of nanoparticles from the two incubation conditions. Images of the slabs were taken initially, at 1.5 hours and after overnight incubation it was apparent that the neutral pH caused the DMA hydrogel to degrade rapidly, thereby releasing the nanoparticles into the bath. The hydrogel that was placed in the acidic bath remained intact for an additional 2 weeks of follow up. Apart from protecting bioactive compounds from the destructive effects of acids in the stomach, the present invention relates to microencapsulation that enhances the bioavailability of bioactive substances.

Modification of alginate by attachment of dopamine molecules also resulted in enhanced adhesiveness of the alginate polysaccharide. Thus, in an embodiment, it is contemplated and therefore within the scope of the invention that stable microbeads of the modified alginate might potentially be used to delay the transit time of the beads containing therapeutic bioactive compounds in the intestine such that sustained delivery of the compounds would be achieved for enhanced therapeutic efficacy. It was found that the stability of the modified alginate microbeads can be achieved by increasing the degree of modification of alginate with dopamine.

### Procedures for Encapsulating Various Bioactive Substances

The following are examples of procedures used to micro-encapsulate certain identified bioactive substances with the modified alginate. These examples are illustrative only and are not to be considered the only embodiments of the invention.

### Alginate Micro-Encapsulation Procedures

The following procedures can be used to micro-encapsulate any of a plurality of bioactive substances.

Modified alginate is dissolved in Hanks Balanced Salt Solution (HBSS) (Sigma) overnight at 4° C. The desired compounds, drugs, or cells are suspended in the alginate and mixed to ensure uniform distribution of the various substances. The suspensions are then loaded into a peristaltic pump, extruded through a 15-gauge needle at a rate of 0.2 ml/min, and droplets of the suspension are received in a bath of calcium chloride (CaCl₂) for crosslinking (gelation). The crosslinked microbeads are then collected and washed twice with HBSS supplemented with 25 mM CaCl₂.

### Effects of Alginate Modification

There exist two methods (described below) that can be used for modifying the alginate, each of which determines how the alginate will degrade in more neutral pH solutions. A first method for modifying the alginate degrades slower relative to a second method for modifying the alginate, which can be useful for targeted delivery in the gastrointestinal tract (GIT). The second method has been shown to result in alginate microbeads that fall apart (degrade) within 30 minutes, while microbeads generated from alginate modified by the first method degrade in about 1-2 hours. The two methods are as follows:

### Method 1:

Alginate (I mmol equivalent) is dissolved in about 25 mL of premade phosphate buffer solution (pH 6.0) and 25 mL of acetonitrile. 1.1 mol equivalent of EDC (1-Ethyl-3-(3-dimethylaminopropyl)carbodiimide) and 1.5 mol equivalent of NHS (N-Hydroxysuccinimide) are added to the solution. The reaction is stirred for 1 h in the dark followed by addition of 1.8 equivalent of 4(2-ethylamino) benzoic acid.HCl. The mixture is stirred in the dark under inert atmosphere for the next 18 h. The solution is then dialyzed for 12 h in 0.1M NaCl and then deionized water for 24 h. The solvent is then removed via lyophilization. 210 mg of white, cotton textured modified alginate material was obtained.

### Method 2:

The alginate (1 mmol equivalent) is dissolved in ultrapure water (Millipore Sigma) with 10% (v/v) isopropanol to about 8 mg/mL. The solution is degassed with N₂ and chilled to about 2-4° C. A degassed solution of sodium (meta)periodate (0.25M solution) is added based on the desired degree of oxidation intended (at 0.5% oxidation). The mixture is stirred for 48 h in the dark and then dialyzed in ultrapure water until the conductivity was below 2 μS and then dried via lyophilization.

The periodate oxidized alginate is dissolved in ultrapure water and methanol (12% v/v). Equivalent moles of amine substituent are added to the solution matching the % oxidized alginate and about 10 mol equivalent of pic-BH₃ (2-picoline-borane). The pH of the mixture is adjusted to about pH 6 using phosphate buffer and the solution is stirred in the dark for 24 h. The sample is dialyzed in 0.1 M NaCl for 12 h followed by dialysis in ultrapure water for 24 h and then lyophilized.

Substituent presence and modification quantification was done via quantitative ¹H NMR using 3-(trimethylsilyl)-2,2,3,3-tetradeuteratedpropionic acid sodium salt (TMSP-d4) as an internal standard.

Diffusion-Ordered Spectroscopy (2D-DOSY—Linear gradient) was acquired to confirm the covalent bonding of the substituent to the alginate polymer.

### Additional Modifications

Two additional parameters can be changed to further modify the degradation rate of the modified alginate. They are: (1) the type ofalginate used, and (2) the alginate concentration. Both LVM (low viscosity mannuronic acid) and LVG (low viscosity guluronic acid) alginate are commonly used for encapsulation, but LVG creates a stronger hydrogel network which slows down the degradation of the modified alginate. Also, increasing the concentration of alginate creates a denser network which slows down the degradation rate. Each of these variables can be adjusted or combined to create a targeted delivery system for the desired compound depending on the mammalian species involved and where and when to deliver the compound of interest.

The following more specific examples are illustrative of several of the useful embodiments of the present invention.

### Procedures for Micro-Encapsulating Dewormer Drugs/Medications

As one example of micro-encapsulating dewormer medications for horses, cattle, sheep, dogs, cats, and other animals, the equine deworming drug Benzimidizole can be micro-encapsulated in the Modified Alginate. Benzimidizole is approximately 118.14 g/mol. in powder form. The procedure is as follows.

The Benzimidizole should be mixed with the Modified Alginate at approximately 20% w/v (20 g/100 mL). The suspension can then be loaded into a peristaltic pump, extruded through a 15-gauge needle at a rate of approximately 0.2 mL/min, and droplets of the suspension are received in a bath of calcium chloride (CaCl₂) for crosslinking (gelation). The crosslinked microbeads are then collected and washed twice with HBSS (Hanks Balanced Salt Solution) supplemented with 25 mM CaCl₂. There will be approximately 1.693*10⁻³ mol of Benzimidizole in the 1 mL of alginate.

By encapsulating approximately 0.2 g of powder per 1 mL of the Modified Alginate, approximately 40 micro-beads containing the specified amount of Benzimidizole are produced.

### Procedures for Micro-Encapsulating ADHD Medications

As one example of micro-encapsulating ADHD medications, the medication Methylphenidrate can be micro-encapsulated in the Modified Alginate. Methylphenidrate has a molecular weight of 233.31 g/mol in powder form. The procedure is as follows.

The Methylphenidrate is mixed with the alginate at 20% w/v (20 g/100 mL). The suspension is then loaded into a peristaltic pump, extruded through a 15-gauge needle at a rate of 0.2 mL/min, and droplets of the suspension are received in a bath of calcium chloride (CaCl₂) for crosslinking (gelation). The crosslinked microbeads will then be collected and washed twice with HBSS supplemented with 25 mM CaCl₂. There will be approximately 8.572*10⁻³ mol of Methylphenidrate in the 1 mL of alginate.

### Procedures for Micro-Encapsulating Prescription Pain Medications

As one example of micro-encapsulating prescription pain medications, Oxycodone can be micro-encapsulated in the Modified Alginate. Oxycodone has a molecular weight of 315.364 g/mol in powder form. The procedure is as follows.

The Oxycodone can be mixed with the alginate at 20% w/v (20 g/100 mL). The suspension is then loaded into a peristaltic pump, extruded through a 15-gauge needle at a rate of 0.2 mL/min, and droplets of the suspension are received in a bath of calcium chloride (CaCl₂) for crosslinking (gelation). The crosslinked microbeads will then be collected and washed twice with HBSS supplemented with 25 mM CaCl₂. There will be approximately 6.34*10⁻⁴ mol of Oxycodone in the 1 mL of alginate.

### Procedures for Micro-Encapsulating Probiotics

As one example of micro-encapsulating probiotics, the probiotic Lactobacillus Casei NCDC 298 can be micro-encapsulated in the Modified Alginate. The procedure is as follows.

The Lactobacillus is cultured overnight in MRS broth then spun down and mixed with the alginate. The suspension is then loaded into a peristaltic pump, extruded through a 15-gauge needle at a rate of 0.2 ml/min, and droplets of the suspension are received in a bath of calcium chloride (CaCl₂) for crosslinking (gelation). The crosslinked microbeads are then collected and washed twice with HBSS supplemented with 25 mM CaCl₂. There will be approximately 40.0*10⁹ lactobacilli in the 1 mL of alginate.

### Procedures for Micro-Encapsulating Dietary Supplements

As one example of micro-encapsulating dietary supplements, Vitamin E (alpha-tocopherol acetate) can be micro-encapsulated in the Modified Alginate. Alpha-tocopherol acetate has a molecular weight of 472.743. The procedure is as follows.

The alpha-tocopherol acetate is mixed with the alginate at 20% w/v (20 g/100 mL). The suspension is then loaded into a peristaltic pump, extruded through a 15-gauge needle at a rate of 0.2 mL/min, and droplets of the suspension are received in a bath of calcium chloride (CaCl₂) for crosslinking (gelation). The crosslinked microbeads will then be collected and washed twice with HBSS supplemented with 25 mM CaCl₂. There will be approximately 4.23*10⁻⁴ mol of Vitamin E in the 1 mL of alginate.

### Procedures for Micro-Encapsulating Selective Serotonin Reuptake Inhibitors (SSRIs)

As one example of micro-encapsulating Selective Serotonin Reuptake inhibitors, Paroxetine (Paxil) can be micro-encapsulated in the Modified Alginate. Paroxetine has a molecular weight of 374.83 g/mol in powder form. The procedure is as follows.

The Paroxetine is mixed with the alginate at 20% w/v (20 g/100 mL). The suspension is then loaded into a peristaltic pump, extruded through a 15-gauge needle at a rate of 0.2 mL/min, and droplets of the suspension are received in a bath of calcium chloride (CaCl₂) for crosslinking (gelation). The crosslinked microbeads will then be collected and washed twice with HBSS supplemented with 25 mM CaCl₂. There will be approximately 5.33*10⁻⁴ mol of Paroxetine in the 1 mL of alginate.

### Procedures for Micro-Encapsulating Non-Prescription Pain Medications

As one example of micro-encapsulating non-prescription pain medications, Acetylsalicylic Acid (Aspirin) can be micro-encapsulated in the Modified Alginate. Acetylsalicylic Acid has a molecular weight of 180.157 g/mol in powder form. The procedure is as follows.

Acetylsalicylic Acid is mixed with the alginate at 20% w/v (20 g/100 mL). The suspension is then loaded into a peristaltic pump, extruded through a 15-gauge needle at a rate of 0.2 mL/min, and droplets of the suspension are received in a bath of calcium chloride (CaCl₂) for crosslinking (gelation). The crosslinked microbeads will then be collected and washed twice with HBSS supplemented with 25 mM CaCl₂. There will be approximately 1.11*10₋₃ mol of Acetylsalicylic Acid in the 1 mL of alginate.

Thus, in an embodiment the present invention relates to an oral delivery system for medicines and other substances in humans and other animals. The method is advantageous that it provides: (1) protection of the encapsulated substance from destruction or degradation by stomach acids and enzymatic action in the stomach, (2) the elimination of stomach upset, nausea, and vomiting caused by certain medicines and other substances when they are introduced into the stomach, (3) a resulting increase in the bioavailability of the substance when it reaches the small intestine, where the contents of the micro-capsule are released into the small intestine through the process of diffusion, or its contents are fully released when the micro-capsule breaks apart in the small intestine due to the PH differential between the stomach and the small intestine, (4) a reduction in the dosage required, since the overall bioavailability of the substance has been increased, (5) the ability to control the rate of release of the substance as it passes through the small intestine by adjusting the chemistry of the aromatic/carbohydrate combination, thereby increasing or decreasing the sensitivity of the micro-capsule material to the PH differential, (6) the ability to completely hide or mask the real taste or flavor of the medicine or substance in the micro-capsule for easier administration of unpleasant or noxious tasting substances to humans and animals, (7) the ability to add the micro-encapsulated substance to existing desirable foods or “treats,” so the medicine or other substance can be consumed readily by humans or animals undetected, and (8) the ability to suspend the micro-encapsulated substance in liquids for easier administration to humans (particularly children) and certain animals.

### Uses of the Invention

The present invention has a plurality of uses including the ability to deliver medicines, drugs, chemicals, proteins, enzymes, probiotics, dietary supplements, and other bioactive substances to humans and animals, which can be used to treat, and/or inhibit diseases, parasites, and other conditions in humans and animals, eliminate or reduce pain associated with a wide variety illnesses, diseases and conditions, and maintain the good health and well-being of humans and animals, including, but not limited to, the following classes or categories of medicines and other bioactive substances:
(a) Oral agents including sulfonylureas, insulin-sensitizers, and insulin;
(b) Anticancer drugs and chemotherapeutic agents;
(c) Neuroleptics and antipsychotic drugs, tranquilizers, antidepressants and sedatives;
(d) Antibiotics and antimicrobials;
(e) Antiepileptic and anticonvulsant drugs;
(f) Neurotransmitters;
(g) Anti-hypertensives such as beta blockers and ACE-inhibitors; and
(h) Statins including Lipitor and Zocor, among others.

### Pain Medications

The invention can be used to eliminate or reduce pain and inflammation by administering one or more of the following classes of pain medications to humans and animals without encountering certain negative side effects:

1. Non-prescription pain medications, such as nonsteroidal anti-inflammatory drugs, including, but not limited to, aspirin (acetylsalicylic acid), ibuprofen, naproxen, and any combinations thereof.

2. Prescription pain medications, such as nonsteroidal anti-inflammatory drugs, including, but not limited to, fenoprofen, flurbiprofen, ketoprofen, oxaprozin, diclofenac sodium, etodolac, indomethacin, ketorolac, sulindac, tolmetin, meclofenamate, mefenamic acid, nabumetone, piroxicam, and any combinations thereof.

3. Prescription pain medications, such as opioid drugs, including, but not limited to, codeine, fentanyl, hydrocodone, hydrocodone with acetaminophen, hydromorphone, meperidine, methadone, morphine, oxycodone, tapentadol, oxymorphone, buprenorphine, tramadol, oxycodone with acetaminophen, naloxone, and any combinations thereof.

### Probiotics

The invention can be used to deliver live probiotics and other beneficial micro-organisms to humans and animals for their therapeutic benefits, such as:

1. Probiotic strains and other micro-organisms found in or beneficial to the human microbiome, including, but not limited to:
(a) Probiotic Strains of the Lactobacillus species of bacterium, including, but not limited to, L. acidophilus, L. fermentum, L. plantarum, L. rhamnosus, L. salivarius, L. paracasei, L. gasseri, L. brevis, L. bulgaricus, L. caucasicus, L. helveticus, L. lactis, L. casei, and L. reuteri, and any combination thereof.
(b) Probiotic Strains of the Bifidobacterium species of bacterium, including, but not limited to, B. bifidum, B. longum, and B. infantis, and any combination thereof.
(c) Probiotic strains of the Bacillus species of bacterium, including, but not limited to, B. coagulans, and any combination thereof.
(d) Probiotic strains of the Streptoccocus species of bacterium, including, but not limited to, S. salivarius K12, and S. Salivarius M18, and any combination thereof.
(e) Other probiotic strains of bacterium found in or beneficial to the human microbiome, including, but not limited to, probiotic strains of bacterium used to treat or combat clostridium difficile (c. diff.), as well as other intestinal diseases or conditions, and any combination thereof.

2. Probiotic strains of bacterium found in or beneficial to the microbiome of other animals, including, but not limited to, dogs, cats, horses, cattle, sheep, pigs, chickens, and includes any combination of those probiotic strains of bacterium.

### Dietary Supplements

The invention can be used to deliver vitamins, minerals, micro-nutrients, and other dietary supplements to humans and animals protected from oxidation and without certain negative side effects, such as:

1. Dietary supplements, including, but not limited to, omega-3 fatty acids (EPA/DHA), vitamin D, vitamin B1, B2, B3, B5, B6, B7, B9, B12, B17, vitamin B complex, alpha lipoic acid, and Coenzyme Q10, among others, and any combinations thereof.

### Equine Dewormers

In an embodiment, the invention can be used to deliver deworming medicines and other bioactive substances to horses, such as:

1. Medicines and other bioactive substances used to treat, prevent, inhibit, and/or remove gastrointestinal parasites in horses, such as Strongyles (blood or red worms, including S. vulgaris, S. edentates, and S. equinus), Ascarids (roundworms), Tapeworms, and Bots. These medicines and other bioactive substances include, but are not limited to, Benzimidazoles (including the generics Fenbendazole and Oxibendazole), Macrocyclic Lactones (including the generics Ivermectin and Moxidectin), Tetrahydropurimidines (including the generics Pyrantel Pamoate and Pyrantel Tatrate), and Isquinoline-pyrozines (including the generic Praziquantel), and any combinations thereof.

2. Medicines and other bioactive substances used to treat, prevent, and/or inhibit other diseases and conditions in horses, reduce pain and inflammation, and maintain their general health and well-being.

### Feline and Canine Dewormers

In an embodiment, the invention can be used to deliver deworming medicines and other bioactive substances to dogs and cats, such as the delivery of:

1. Medicines and other bioactive substances used to treat, inhibit, and/or remove gastrointestinal parasites in cats, including, but not limited to, Piperazine, Praziquantel, Ivermectin, Selamectin, Imidacloprid, Moxidectin, and any combination thereof.

2. Medicines and other bioactive substances used to treat, inhibit, and/or remove gastrointestinal parasites in dogs, including, but not limited to, Pyrantel pamoate, Praziquantel, Fenbendazole, Ivermectin, Milbemycin oxime, Selamectin, Imidacloprid, Moxidectin, Spinosad, and any combination thereof.

3. Medicines and other bioactive substances used to treat, prevent, and/or inhibit other diseases and conditions in cats and dogs, reduce pain and inflammation, and maintain their general health and well-being.

### Dewormers Used in Other Animals

In an embodiment, the invention can be used to deliver deworming medicines to other animals, such as:

1. Medicines and other bioactive substances used to treat, inhibit, and/or remove gastrointestinal parasites in other animals, such as cattle, sheep, and pigs, including, but not limited to, Fenbendazole, Ivermectin, Levamisole, Morantel tartrate, Thiabendazole, Albendazole, Oxfendazole, and any combination thereof.

Treating and/or Inhibiting Diseases and Conditions in Other Animals

In an embodiment, the invention can be used to deliver medicines and other bioactive substances to animals such as:

1. Medicines and other bioactive substances used to remove parasites, treat, prevent, and/or inhibit diseases and other conditions, reduce pain or inflammation, and maintain their general health and well-being.

### Rodenticides

In an embodiment, the invention can be used to deliver rodenticides to rodents such as:

1. Chemicals, drugs, compounds, and other substances used to eliminate and/or control rodents or rodent populations, including, but not limited to, Warfarin, Chlorphacinone, Diphacinone, Bromadiolone, Difethialone, Brodifacoum, Bromethalin, Cholecalciferol, Zinc phosphide, Strychnine, triptolide, 4-vinylcyclohexene diepoxide, diterpenoid epoxides, ovotoxins, diterpenoid epoxides, and any combinations thereof.

It is contemplated and therefore within the scope of the invention to include reasonable modifications to the embodiments described above without departing from the spirit and scope of the invention. For example, it is contemplated and therefore within the scope of the invention that any one or more feature(s) that is/are described herein can be combined with any other one or more compatible feature(s) irrespective of the fact that those features may not be mentioned with the same product. Features that are disclosed above as being part of a composition may be incorporated into the methods of the present invention and, similarly, the method steps may be incorporated into the composition. When a range is given, it is contemplated that any subrange that fits within the scope of that range is contemplated as a possible range of the present invention even if the one or more endpoints (which fit within the range) is not disclosed herein. Moreover, it is contemplated and therefore within the scope of the invention that when Markush groups or other list of elements/substituents are given that any subset of those elements/substituents can be used to generate a sub-group. In any event, the present invention is to be described by the below claims.

We claim:

1. A composition comprising a substance wherein said substance comprises one or more (i) drugs, medicines, enzymes, proteins, hormones, vaccines, vitamins, minerals, micronutrients and/or other dietary supplements, (ii) probiotics and/or other microorganisms, (ii) cells, cell parts, and/or other biological materials, and/or (iii) other bioactive compounds or substances,
in combination with a modified alginate, wherein said modified alginate comprises an alginate backbone that has been modified by the addition of an aromatic compound substituent.

2. The composition of claim 1 wherein the aromatic substituent comprises one or more of a dopaminic substituent, a phenolic substituent, a benzoic acid substituent, an anilinic substituent, a toluenic substituent, an amino sulfonamidic benzene substituent and/or mixtures thereof.

3. The composition of claim 2, wherein the aromatic substituent is selected from the group consisting of a 4(2-ethylamino)benzoic acid substituent, a 4(2-ethylamino)phenolic substituent, a 4(2-ethylamino)anilinic substituent, a para (2-ethylamino)toluenic (i.e., (2-ethylamino)4-methylbenzene) substituent, a 4-aminomethyl benzene sulfonamide substituent, a 4-aminoethyl benzene sulfonamide substituent, and mixtures thereof.

4. The composition of claim 3, wherein the composition comprises one or more bioactive substances encapsulated by the modified alginate.

5. The composition of claim 4, wherein the one or more bioactive substances encapsulated by the modified alginate further comprise one or more of
(a) sulfonylureas, insulin-sensitizers, or insulin;
(b) Anticancer drugs or chemotherapeutic agents;
(c) Neuroleptics, antipsychotic drugs, tranquilizers, antidepressants or sedatives;
(d) Antibiotics or antimicrobials;
(e) Antiepileptic or anticonvulsant drugs;
(f) Neurotransmitters;
(g) Anti-hypertensives;
(h) Statins;
(i) Non-prescription pain medications; or combinations thereof;
(j) Prescription pain medications selected from the group consisting of fenoprofen, flurbiprofen, ketoprofen, oxaprozin, diclofenac sodium, etodolac, indomethacin, ketorolac, sulindac, tolmetin, meclofenamate, mefenamic acid, nabumetone, piroxicam, or combinations thereof;
(k) Prescription pain medications selected from the group consisting of codeine, fentanyl, hydrocodone, hydrocodone with acetaminophen, hydromorphone, meperidine, methadone, morphine, oxycodone, tapentadol, oxymorphone, buprenorphine, tramadol, oxycodone with acetaminophen, naloxone, and/or combinations thereof;
(l) Probiotic strains of Lactobacillus species of bacterium selected from the group consisting of Lactobacillus acidophilus, Lactobacillus fermentum, Lactobacillus plantarum, Lactobacillus rhamnosus, Lactobacillus salivarius, Lactobacillus paracasei, Lactobacillus gasseri, Lactobacillus brevis, Lactobacillus bulgaricus, Lactobacillus caucasicus, Lactobacillus helveticus, Lactobacillus lactis, Lactobacillus casei, and Lactobacillus reuteri, and any combination thereof;
(m) Probiotic strains of Bifidobacterium species selected from the group consisting of Bifidobacterium bifidum, Bifidobacterlum longum, Bifidobacterium infantis, and combinations thereof;
(n) Probiotic strains of Bacillus coagulans;
(o) Probiotic strains of Streptococcus species of bacterium selected from the group consisting of Streptococcus salivarius K12, and Streptococcus Salivarius M18, and combinations thereof;
(p). Vitamins, minerals, micro-nutrients, and dietary supplements selected from the group consisting of omega-3 fatty acids (EPA/DHA), vitamin D, vitamin B1, B2, B3, B5, B6, B7, B9, B12, B17, vitamin B complex, alpha lipoic acid, and Coenzyme Q10, and combinations thereof;
(q) Medicines and bioactive substances used to treat Strongyles, Ascarids, Tapeworms, and Bots in horses, wherein said medicines and bioactive substances are selected from the group consisting of Benzimidazoles selected from the group consisting of Fenbendazole and Oxibendazole, Macrocyclic Lactones selected from the group consisting of Ivermectin and Moxidectin, Tetrahydropurimidines selected from the group consisting of Pyrantel Pamoate and Pyrantel Tatrate, and Isquinoline-pyrozines, and combinations thereof;
(r) Medicines and bioactive substances used to treat other diseases or conditions in horses, reduce pain and inflammation, and maintain their general health and well-being;
(s) Medicines and bioactive substances used to treat gastrointestinal parasites in cats selected from the group consisting of Piperazine, Praziquantel, Ivermectin, Selamectin, Imidacloprid, Moxidectin, and combinations thereof;
(t) Medicines and bioactive substances used to treat gastrointestinal parasites in dogs selected from the group consisting of Pyrantel pamoate, Praziquantel, Fenbendazole, Ivermectin, Milbemycin oxime, Selamectin, Imidacloprid, Moxidectin, Spinosad, and combinations thereof;
(u) Medicines and bioactive substances used to treat other diseases and conditions in cats and dogs, reduce pain and inflammation, and maintain their general health and well-being;
(v) Medicines and bioactive substances used to treat gastrointestinal parasites in other animals, selected from the group consisting of Fenbendazole, Ivermectin, Levamisole, Morantel tartrate, Thiabendazole, Albendazole, Oxfendazole, and combinations thereof;
(w) Chemicals, drugs, compounds, and other substances used to control rodents or rodent populations, selected from the group consisting of Warfarin, Chlorphacinone, Diphacinone, Bromadiolone, Difethialone, Brodifacoum, Bromethalin, Cholecalciferol, Zinc phosphide, Strychnine, triptolide, 4-vinylcyclohexene diepoxide, diterpenoid epoxides, ovotoxins, diterpenoid epoxides, and combinations thereof.

6. The composition of claim 1, wherein the modified alginate is stable under acidic conditions but is labile under basic conditions.

7. The composition of claim 6, wherein the modified alginate is stable at a pH of between about 1.5 to 3.5 but is labile when the pH increases to a level above 7.

8. The composition of claim 1, wherein the aromatic substituent is dopamine or 4(2-ethylamino)benzoic acid and an amount of dopamine or 4(2-ethylamino)benzoic acid present in the modified alginate is between about 5% and 15% by weight dopamine or 4(2-ethylamino)benzoic acid.

9. A method of making proteins, micronutrients, dietary supplements and/or probiotics more bioavailable to an individual in need of said proteins, micronutrients, dietary supplements and/or probiotics by administering to said individual said dietary supplements and/or probiotics encapsulated in a modified alginate, said modified alginate being modified by the incorporation of covalently linked dopamine, 4(2-ethylamino)benzoic acid, 4-aminomethyl benzene sulfonamide substituents, or 4-aminoethyl benzene sulfonamide substituents.

10. The method of claim 9, wherein an amount of dopamine, 4(2-ethylamino)benzoic acid, 4-aminomethyl benzene sulfonamide, or 4-aminoethyl benzene sulfonamide present in the modified alginate is between about 5% and 15% by weight dopamine.

11. The method of claim 10, wherein the amount of dopamine, 4(2-ethylamino)benzoic acid, 4-aminomethyl benzene sulfonamide, or 4-aminoethyl benzene sulfonamide present in the modified alginate is between about 8% and 15% by weight dopamine.

12. The method of claim 11, wherein the modified alginate is stable at a pH of around about 3 to 5 and labile at a pH of above 7.

13. A method of preparing an aromatic alginate, said method comprising reacting an alginate with an aromatic substituent, said aromatic substituent comprising one or more of a dopaminic substituent, a 4(2-ethylamino)phenolic substituent, a 4(2-ethylamino)benzoic acid substituent, a 4(2-ethylamino)anilinic substituent, a 4(2-ethylamino)toluenic substituent, a 4-aminomethyl benzene sulfonamide, a 4-aminoethyl benzene sulfonamide or mixtures thereof.

14. The method of claim 13, wherein said method incorporates a dopaminic substituent by reacting alginate with said dopaminic substituent in the presence of one or more of N-Hydroxysuccinimide (NHS) and 1-Ethyl-3-(3-dimethylaminopropyl)carbodiimide.

15. A method of delivering a protein, micronutrient, dietary supplement or probiotic to an individual in need thereof, said method comprising administering to said individual a composition that comprises a modified carbohydrate, said modified carbohydrate comprising a modified alginate, that has been modified by an aromatic substitute, said aromatic substituent comprising one or more of a dopaminic substituent, a phenolic substituent, a benzoic acid substituent, an anilinic substituent, an amino sulfonamide benzene substituent or mixtures thereof.

16. The method of claim 15, wherein the protein, micronutrient, dietary supplement or probiotic is encapsulated by the modified carbohydrate.

17. The method of claim 16, wherein the modified carbohydrate is modified alginate.

18. The method of claim 17, wherein the modified alginate is modified by covalent addition of dopaminic substituents.

19. The method of claim 18, wherein the modified alginate contains dopaminic substituents in an amount of about 5 to about 15% by weight dopamine.

20. The method of claim 18, wherein the modified alginate is stable at a pH of about 3 to 5.
